(19) United States
(12) Patent Application Publication
(10) Pub. No.: US 2016/0318828 A1
(43) Pub. Date: Nov. 3, 2016

(54) Catalytic Alkane Dehydrogenation

(71) Applicant: ExxonMobil Chemical Patents Inc., Baytown, TX (US)

(72) Inventors: Seth M. Washburn, Houston, TX (US); John S. Coleman, Houston, TX (US); John S. Buchanan, Flemington, NJ (US); Neeraj Sangar, Houston, TX (US); Paul F. Keusenkothen, Houston, TX (US)

(21) Appl. No.: 15/094,279

(22) Filed: Apr. 8, 2016

Related U.S. Application Data
(60) Provisional application No. 62/154,977, filed on Apr. 30, 2015.

(30) Foreign Application Priority Data
May 29, 2015 (EP) 15169871.9

(51) Int. Cl. C07C 5/333

(52) U.S. Cl. CPC C07C 5/3335

(57) ABSTRACT

The invention relates to catalytic alkane dehydrogenation, to olefin produced by catalytic alkane dehydrogenation, and to processes, compositions, process configurations, equipment, and systems useful for carrying out catalytic alkane dehydrogenation. The catalytic alkane dehydrogenation is carried out in a substantially-isothermal reaction zone, which includes at least one active material having catalytic alkane dehydrogenation activity.

### CROSS-REFERENCE OF RELATED APPLICATIONS

This invention claims priority to and the benefit of U.S. Patent Application Ser. No. 62/154,977, filed Apr. 30, 2015, and European Patent Application No. 15169871.9, filed May 29, 2015, the disclosures of which are incorporated by their reference in their entireties. This application is related to U.S. Ser. No. 14/469180 filed Aug. 26, 2014, (attorney docket number 2014EM206).

### FIELD OF THE INVENTION

The invention relates to catalytic alkane dehydrogenation, and more particularly to olefin produced by catalytic alkane dehydrogenation. The invention encompasses processes, compositions, process configurations, equipment, and systems useful for carrying out catalytic alkane dehydrogenation. The catalytic alkane dehydrogenation is carried out in a substantially-isothermal reaction zone, which includes at least one active material having catalytic alkane dehydrogenation activity.

### BACKGROUND OF THE INVENTION

Olefin, also called alkene, is a class of unsaturated hydrocarbon compounds containing at least one pair of carbon atoms, the carbon atoms of the pair being directly linked by a double bond. Since the double-bonded carbons allow the compounds to be reacted with a wide variety of other compounds to produce a wide array of useful products, olefin compounds are of considerable commercial importance.

Olefin is produced commercially by a variety of processes, e.g., steam cracking and fluid catalytic cracking of hydrocarbons. Since these processes produce olefin having a range of molecular weights, and typically also other (non-olefin) products, technologies such as catalytic dehydrogenation technologies have been developed to yield a particular olefin-an olefin having a particular number of carbon atoms. Catalytic hydrogenation involves catalytically reacting (i.e., dehydrogenating) a saturated hydrocarbon compound such as alkane to produce the desired olefin. For example, catalytic dehydrogenation technologies include those which catalytically react propane to produce propene.

Jesper et al., Catalytic Dehydrogenation of Light Alkanes on Metals and Metal Oxides, Chem. Rev., 2014, 114 (20), pp 10613-10653, describe a number of commercial dehydrogenation processes. Such processes include the Catofin Process (Lummus), Oleflex Process (UOP), Steam Active Reforming (STAR) Procss (Uhde), Fluidized Bed Dehydrogenation (FBD) Process (Snamprogetti and Yarsintez) and Linde-BASF Isothermal Fixed Bed Process.

Some of these are fixed-bed processes. For example, Catofin, which is based on the Houdry Catadiene process, for isobutane dehydrogenation to isobutene, dehydrogenates propane in 5-8 parallel adiabatic fixed bed reactors containing a chromia-alumina catalyst. Process conditions include a temperature of approximately 575° C. and a pressure between 0.2 and 0.5 bar. Another fixed-bed process, STAR, operates at a pressure of 6 to 9 bar and a temperature of between 500° C. to 600° C. Steam is added to the alkane feed to reduce alkane partial pressure, resulting in less coke formation. The feed is conducted to a first reactor, which contains a catalyst comprising Pt—Sn supported on a (basic) zinc-aluminate. A calcium/magnesium-aluminate binder is used to stabilize the catalyst in the presence of the steam. The gas mixture exiting the first reactor is cooled prior to being introduced into the second reactor (called an oxyreactor), where an oxygen-steam mixture is used to selectively combust part of the hydrogen formed during the dehydrogenation. Combusting the hydrogen shifts equilibrium toward higher olefin yields. Like STAR, The Linde-BASF Dehydrogenation Process is a fixed bed reactor process, using an alkane feed diluted with steam. The catalyst comprises Pt—Sn supported on ZrO₂. The process is carried out isothermally at a temperature of 590° C. Isothermal conditions are maintained by externally heating the reactor.

Other processes utilize a fluidized bed. For example, Oleflex utilizes Pt—Sn-based catalyst in a fluidized bed operating at pressures between 1 and 3 bar and a temperature of 525° C. to 705° C. Polymer-grade propylene from the reaction product by recovering C₃ hydrocarbon in a de-ethanizer, and then separating propylene from unreacted propane in a splitter. Another fluidized-bed process, FBD, incorporates the use of a fluid catalytic cracking reactor system. The alkane feed flows through a staged fluidized bed reactor, contacting the alkane with heated CrOₓ/Al₂O₃ catalyst, promoted with an alkali metal. The alkane dehydrogenation is carried out at a pressure of 1.1 to 1.5 bar and a temperature of 550° C. to 600° C. Carbon deposits formed on the catalyst during the dehydrogenation, resulting in deactivated catalyst. The deactivated catalyst is transported to a regenerator connected to the reactor to combust the carbon deposits, reactivating the catalyst for reuse. Fuel gas is added to the regenerator to provide sufficient heat to carry out regeneration. The regenerated catalyst is returned to the fluidized bed reactor at a temperature of around 560° C., which is sufficient to carry out the desired dehydrogenation reaction.

Since alkane catalytic dehydrogenation is an equilibrium reaction, process conditions affect the amount of olefin that can be produced. An example of such a reaction is the dehydrogenation of propane to produce propene:

C₃H₈⇄C₃H₆+H₂(ΔH⁰₂₉₈=124.3 kJ mol⁻¹)

The above reaction shows that heat (124.3 kJ) is required to dehydrogenate propane, which means that the reaction is an endothermic reaction. Propylene is the desired product of the reaction, with hydrogen being an undesirable byproduct—the presence of hydrogen in the reaction product results in converting propylene back to propane. Typically, dehydrogenation of C₂-C₄ alkane to produce C₂-C₄ olefin requires a reaction temperature in the range of 550° C. to 750° C., with the conversion of the alkane being about 50% at about 1 bar.

According to Le Chatelier's principle, higher conversions of alkane to olefin can be obtained by increasing the reaction temperature and/or reducing the hydrogen gas (molecular hydrogen) partial pressure. However, raising the temperature can result in additional undesirable byproducts being produced through one or more undesired hydrogenolysis, cracking and isomerization side reactions, e.g., increased feed cracking to methane, etc.

Molecular hydrogen partial pressure can be lessened by removing hydrogen molecules produced by the dehydrogenation reaction through selective hydrogen combustion (SHC). One way to do this is disclosed in de Graaf et al., Two-Step Catalytic Oxidative Dehydrogenation of Propane: An Alternative Route to Propene, Vol. 9, No. 4, 397-403, 2005, Organic Process Research & Development. This reference discloses selectively combusting molecular hydrogen produced during the dehydrogenation reaction using oxygen removed from a solid oxygen carrier (a “SOC”; also called an oxygen storage material). The SOC is mixed with the dehydrogenation catalyst. During the combustion, oxygen from the SOC reacts with the molecular hydrogen from the dehydrogenation to produce water.

SOCs are typically porous, having a pore size which is large enough to admit molecular hydrogen, but small enough to exclude the relatively large alkane and olefin molecules. At the start of the dehydrogenation, the SOC is in a state identified as “SOₓC”, indicating that oxygen is available for removal from the SOC. During the dehydrogenation, molecular hydrogen produced by that reaction enters the SOC's pores, where it combusts with the SOC's available oxygen. Since molecular hydrogen produced in the dehydrogenation reaction can more readily migrate into the SOC than can alkane and olefin, equilibrium of the dehydrogenation reaction shifts toward increased olefin production and away from olefin hydrogenation, alkane oxidation, and olefin oxidation. Once the oxygen available in the SOC for combustion is depleted, the SOC will be in a reduced state identified conceptually as “SOₓ₋₁C”. The alkane feed is then stopped, the SOC is re-oxidized from SOₓ₋₁C to an oxidized state that is conceptually identified as “SOₓC,” and the process is repeated. The reduction and oxidation of the SOC are conceptually exemplified by the following equations:

H₂+SOₓC→H₂O+SOₓ₋₁C (Reduction)

O₂+2SOₓ₋₁C→2SOₓC (Oxidation)

Although the de Graaf alkane dehydrogenation process is efficient to the extent that heat for the dehydrogenation reaction is supplied from the highly exothermic combustion of molecular hydrogen, excessive heat is produced in the reactor. At the same time, selectivity toward olefin production remains undesirably low.

What is desired is an alternative process to convert alkane to desired olefin product. Processes for converting alkane to a desired olefin at high conversion of the alkane and high selectivity to the desired olefin are particularly desired.

### SUMMARY OF THE INVENTION

This invention provides an alternative process to convert alkane to desired olefin product. In particular, this invention provides a catalytic process for dehydrogenating alkane to a desired olefin, with the process being carried out at high conversion of the alkane and high selectivity to the desired olefin. The catalytic dehydrogenation process is particularly desirable for converting alkanes such as C₂₊ alkanes into C₂₊ olefin, particularly C₃₊ alkane into C₃₊ olefin. In particular, the process is effective for converting ethane to ethylene, propane to propylene, and butane to butenes.

More particularly, the invention relates to a process for catalytically producing olefin from C₂₊ alkane. The process is carried out in a substantially isothermal reaction zone having an average temperature in the range of from 400° C. to 700° C. At least one active material is located in the reaction zone, the active material having catalytic alkane dehydrogenation functionality at the average temperature.

At the start of the process, flow of alkane-containing feed comprising C₂₊ alkane is established into the reaction zone. At least a portion of the C₂₊ alkane is dehydrogenated to produce a dehydrogenation product comprising olefin and molecular hydrogen. The dehydrogenating is carried out in the reaction zone in the presence of at least a portion of the active material. At least a portion of the dehydrogenation product's molecular hydrogen is combusted in the reaction zone during the catalytic dehydrogenation to produce a combustion product comprising water. The reaction zone is maintained substantially isothermal at the average temperature during the dehydrogenation and combustion. A reaction product is conducted away from the reaction zone, the reaction product comprising at least a portion of the dehydrogenation product's olefin and at last a portion of the combustion product's water.

One way the process differs from conventional processes is that a substantially isothermal temperature profile is maintained in the reaction zone during the dehydrogenation/combustion. Doing so significantly increases the process's selectivity to olefin production, and significantly decreases selectivity to undesirable products like methane and catalyst coke.

In another aspect, the invention relates to an apparatus for catalytically dehydrogenating an alkane-containing feed to produce an olefin-containing product. The apparatus comprises a reactor vessel having an interior volume, and at least one reaction zone within the reactor vessel's interior volume. The reaction zone includes at least one bed of active material comprising dehydrogenation catalyst. At least one inlet conduit in fluidic communication with the reactor vessel's interior volume is used for conveying the alkane containing feed into the reaction zone. The apparatus further comprises at least one heat transfer conduit for bringing a heat transfer fluid into indirect thermal contact with the reaction zone. The heat transfer conduit is substantially closed to flow of the heat transfer fluid into the reactor vessel's interior volume, and is substantially open to the flow of heat between the reaction zone and the heat transfer fluid. The heat transfer conduit is configured to maintain the reaction zone in a substantially isothermal temperature profile at an average temperature 400° C. to 700° C. during the catalytic dehydrogenation. The apparatus further includes at least one outlet conduit in fluidic communication with the reactor vessel's interior volume for conveying the olefin containing product away from reaction zone. In another aspect, the invention relates to a system for carrying out the catalytic dehydrogenation process in the apparatus.

### BRIEF DESCRIPTION OF THE DRAWINGS

FIG. 1 is a simulation of an adiabatic packed bed reactor filled with a mixture of catalyst material comprising i) a first component having propane dehydrogenation (“PDH”) functionality and ii) a second component having selective hydrogen combustion (“SHC”) functionality and an oxygen storage functionality.

FIG. 2 is a simulation of an isothermal packed bed reactor filled with a mixture of catalyst material comprising i) a first component having PDH functionality and ii) a second component having SHC functionality and oxygen storage functionality.

Although the invention can be described in terms of a catalytic dehydrogenation process for producing olefin, particularly a non-oxidative catalytic dehydrogenation process, the invention is not limited thereto. In other words, to the extent that the following detailed description is specific to a particular embodiment or a particular use, this is intended to be illustrative only, and is not to be construed as limiting the scope of the invention. On the contrary, it is intended to cover all alternatives, modifications and equivalents that may be included within the spirit and scope of the invention.

### DETAILED DESCRIPTION OF THE INVENTION

The invention is directed to a catalytic alkane dehydrogenation reaction process for producing olefin. The process is carried out in a reaction zone, which includes at least one active material, the active material having catalytic alkane dehydrogenation functionality and optionally selective hydrogen combustion functionality. At least a portion of the alkane in the feed entering the reaction zone is catalytically dehydrogenated in the presence of at least a portion of the active material, the reaction zone having a substantially isothermal temperature profile at an average reaction zone temperature in the range of from about 400° C. to 700° C. The dehydrogenation reaction produces a dehydrogenation product comprising olefin, hydrogen, and typically further comprising unreacted alkane.

In order to shift equilibrium of the alkane catalytic dehydrogenation reaction toward olefin production, at least a portion of the dehydrogenation product's molecular hydrogen is combusted to produce water. The combustion decreases the amount of molecular hydrogen present in the reaction zone, which shifts equilibrium toward greater olefin production, resulting in a reaction product having a greater olefin concentration relative to that obtained when the dehydrogenation reaction is operated in equilibrium without molecular hydrogen combustion. Typically, the combustion is selective combustion, i.e., one that favors reacting oxygen with molecular hydrogen over reacting oxygen with hydrocarbon (especially alkane and/or olefin). More typically, the combustion is carried out using oxygen made available from at least one SOC, e.g., by utilizing as a multi-component active material having a first component which includes at least one active material having dehydrogenation functionality and a second component which includes at least one SOC. After available oxygen in the SOC is depleted, alkane flow to the reaction zone can be curtailed or halted, which ends catalytic alkane dehydrogenation mode (“dehydrogenation mode”) operation. To at least partially restore the SOC's oxidant content, an oxidant flow is initiated and the process is operated in regeneration mode. Regeneration mode process conditions are regulated in the reaction zone during oxidant flow so that the SOC sorbs at least a portion of the oxidant flow's oxidant, which replenishes the SOC, and optionally to combust any coke that may have accumulated on the active material during prior one or more prior periods of catalytic dehydrogenation. After the SOC is sufficiently replenished with oxidant and sufficient coke has been removed from the active material, the oxidant flow is curtailed or substantially halted. This ends regeneration mode operation. Alkane flow is then re-established into the reaction zone, and reaction zone process conditions are re-set for catalytic alkane dehydrogenation in a substantially isothermal temperature profile. This commences (or re-commences) catalytic alkane dehydrogenation mode.

Since dehydrogenation mode is carried out under substantially isothermal conditions, there is a significantly greater olefin content in the reaction product compared to conventional, non-isothermal alkane dehydrogenation reactions. Maintaining a substantially isothermal temperature profile within the reaction zone lessens formation of undesirable dehydrogenation reaction byproducts, e.g., byproducts resulting from undesirable side reactions. The temperature profile is controlled by moderating heat in the reaction zone during the catalytic dehydrogenation of the feed's alkane and the selective combustion of the dehydrogenation product's molecular hydrogen. A reaction product that is rich in olefin, the olefin-rich product, can be advantageously recovered or removed from the substantially isothermal reaction zone. The olefin-rich product has greater olefin content, lesser unreacted alkane content, and a lesser content of undesired byproducts relative to the reaction product produced under non-isothermal (e.g., adiabatic) conditions.

The process is highly efficient for converting alkane to olefin, such as converting at least one of ethane, propane, butanes, to one or more of ethylene, propylene, and butenes.

The process is also highly energy efficient in that a substantially isothermal temperature profile is at least partially maintained combusting the dehydrogenation product's molecular hydrogen in the vicinity of the catalytic dehydrogenation reaction. This cools those portions of the reaction zone proximate to the combustion and heats those proximate to dehydrogenation, resulting in a shift of the reaction zone's temperature profile toward an isothermal distribution.

During the catalytic alkane dehydrogenation, combustion is distributed in the reaction zone to (i) consume the dehydrogenation product's molecular hydrogen and (ii) transfer heat released by the exothermic combustion to the endothermic dehydrogenation reaction. The first factor increases olefin yield by shifting equilibrium toward olefin production and away from the reverse reaction (olefin hydrogenation to produce alkane). The second factor lessens the effect of undesired side reactions (e.g., cracking and/or coking), which can predominate in higher-temperature regions of the reaction zone. Isothermal operation also results in fewer high-temperature excursions at locations within the reaction zone, especially at locations downstream of alkane dehydrogenation. This feature is used advantageously in certain aspects, e.g., those where it is desirable to recover the reaction product's olefin without energy-intensive cryogenic separation.

Lessening the amount of coke formed during the dehydrogenation also leads to a significant improvement over conventional catalytic alkane dehydrogenation processes. It has been observed that during regeneration mode the rate of coke removal (e.g., by combustion) from the active material is significantly slower than the rate of oxidant replenishment in the SOC. This leads to a loss of the active material's catalytic dehydrogenation activity, resulting primarily from an accumulation of coke on the active material. This problem is worsened when the process is operated cyclically, e.g., by continuously alternating catalytic dehydrogenation and regeneration modes. Conventional methods for overcoming this difficulty, e.g., by (i) lengthening regeneration mode's duration and/or (ii) increasing the amount of oxidant in the oxidant flow during regeneration mode, are complex and inefficient. Lengthening the duration of regeneration mode leads to a loss in olefin yield, and also potentially an irreversible decrease in the active material's catalytic dehydrogenation activity. The activity loss results primarily from a loss of catalyst dispersion. Increasing the oxidant flow's oxidant content desirably shortens the duration of regeneration mode, but further increases the loss of catalyst dispersion. The invention at least partially overcomes these difficulties by increasing the process's selectivity to the desired olefin product and decreasing the process's selectivity for producing catalyst coke. Since less coke is produced on the active material than is the case in conventional catalytic alkane dehydrogenation processes, the duration of regeneration mode can be decreased to the time needed to sufficiently replenish the SOC with oxidant. Should any additional regeneration mode duration be needed for removing sufficient catalyst coke, this additional duration will be much less than that needed in the conventional process because the amount of catalyst coke is much less.

For the purpose of this description and appended claims, the following terms are defined:

The term “Cₙ” hydrocarbon means hydrocarbon having n carbon atom(s) per molecule, wherein n is a positive integer. The term “Cₙ₊” hydrocarbon means hydrocarbon having at least n carbon atom(s) per molecule, wherein n is a positive integer. The term “Cₙ₋” hydrocarbon means hydrocarbon having no more than n number of carbon atom(s) per molecule, wherein n is a positive integer. The term “hydrocarbon” means compounds containing hydrogen bound to carbon, and encompasses (i) saturated hydrocarbon, (ii) unsaturated hydrocarbon, and (iii) mixtures of hydrocarbons, including mixtures of hydrocarbons (saturated and/or unsaturated) having different values of n.

The term “alkane” means substantially saturated compounds containing hydrogen and carbon only, e.g., those containing ≦1% (molar basis) of unsaturated carbon atoms. As an example, the term alkane encompasses C₂ to C₂₀ linear, iso, and cyclo-alkanes.

The term “unsaturate” or “unsaturated hydrocarbon” mean a C₂₊ hydrocarbon containing at least one carbon atom directly bound to another carbon atom by a double or triple bond. The term “olefin” means an unsaturated hydrocarbon containing at least one carbon atom directly bound to another carbon atom by a double bond. In other words, an olefin is a compound which contains at least one pair of carbon atoms, where the first and second carbon atoms of the pair are directly linked by a double bond.

The term “Periodic Table” means the Periodic Chart of the Elements, as it appears on the inside cover of The Merck Index, Twelfth Edition, Merck & Co., Inc., 1996.

The term “reaction stage” or “reactor stage” means at least one dehydrogenation reactor, and optionally including means for conducting one or more feeds thereto and/or one or more products away therefrom. With respect to reactors, the term “zone” or “region” means a location within a reactor, e.g., a specific volume within a reactor and/or a specific volume between two reactors. A “reaction zone” or “reactor zone” is a volume within the reactor for conducting dehydrogenation of alkane. The term “fixed bed catalytic reactor” means a catalytic reactor having at least one bed of catalyst, wherein the catalyst is substantially retained within the bed and remains in a substantially fixed location within the bed.

The term “selectivity” refers to the production or consumption of a specified compound in a catalytic reaction. As an example, the phrase “the reaction has a 100% propylene selectivity” means that the reaction produces a reaction composition comprising 100% propylene. As another example, the phrase “selective hydrogen combustion”, means a reaction in the presence of oxidant, molecular hydrogen, and hydrocarbon which favors molecular hydrogen combustion over hydrocarbon combustion.

A reaction zone is substantially isothermal when it has a substantially isothermal temperature profile over the reaction zone. A substantially isothermal temperature profile is one having a substantially constant temperature over a designated reaction zone. The term “substantially constant temperature” means that the temperature at any location in designated zone is within +/−10° C. of the zone's average temperature. The “average temperature” means the average temperature of the designated reaction zone when the zone is substantially in thermodynamic equilibrium. The average temperature is determined by sampling the zone's temperature a plurality of locations, adding the sampled temperatures, and dividing that sum by the number of sampling locations. Typically, sampling is carried out at three or more locations. The first location is proximate to the place in the reaction zone where alkane is introduced. The third location is proximate to the place in the reaction zone where reaction product is removed. The second location is approximately midway along a direct path from the first location to the third location, from upstream to downstream. Those skilled in the art will appreciate that additional sampling locations may be needed to accurately determine the average reaction zone temperature of large and/or complex reactors. Besides being spatially substantially isothermal, the reaction zone can optionally be temporally substantially isothermal, meaning that the reaction zone's average temperature does not vary by more than +/−10° C. during the time the process is carrying out the specified catalytic alkane dehydrogenation reaction.

The term “non-oxidative dehydrogenation” means a dehydrogenation reaction carried out with little if any oxidative coupling of the alkane molecule to produce olefin. The term “non-oxidative alkane dehydrogenation functionality” refers to an alkane dehydrogenation catalyst material participating in an alkane dehydrogenation reaction in which olefin production occurs predominantly in the absence of forming an oxidized alkane intermediate.

The term “oxygen storage functionality” refers to a material having oxygen sorptive capacity and oxygen release ability for releasing sorbed oxygen. Respecting SOCs, term “oxidant” means any oxygen-bearing material that can be sorbed and released by the SOC for combustion with molecular hydrogen. A material having “selective hydrogen combustion functionality” is one which favors the combustion (oxidation) of molecular hydrogen with sorbed oxygen released from the material over the combustion with the released oxygen with one or more additional compounds such as hydrocarbon.

### Reactor Feed Compositions

The process utilizes a first feed comprising alkane during dehydrogenation mode and optionally a second feed comprising oxidant during an optional regeneration mode. During dehydrogenation mode, alkane in the alkane-containing feed reacts in the presence of an active material having the specified dehydrogenation functionality. The reaction occurs in a substantially isothermal reaction zone, and produces a dehydrogenation product comprising olefin and molecular hydrogen. In order to shift equilibrium toward olefin production and to shift the reaction zone's temperature profile toward an isothermal profile, at least a portion of the dehydrogenation product's molecular hydrogen is combusted. Although any convenient oxidant source can be used for the combustion, the process typically uses sorbed oxygen released from at least one SOC. At the end of dehydrogenation mode, the flow of alkane-containing feed to the reaction zone is curtailed or substantially halted. When a regeneration mode is used, a flow of a second feed comprising oxidant is established to replenish the SOC with oxidant and to remove at least a portion of any catalyst coke which may have accumulated on the active material in the reaction zone during the dehydrogenation. After replenishing a sufficient amount of the SOC's oxidant and removing a sufficient amount of any accumulated coke, the flow of second feed is curtailed or substantially halted, which ends regeneration mode. Alternating dehydrogenation modes and regeneration modes can be carried out continuously. Optionally, additional feeds are used between dehydrogenation mode and regeneration mode, e.g., one or more sweep fluids. Selected feeds will now be described in more detail. The invention is not limited to these feeds, and this description is not meant to foreclose other feeds within the broader scope of the invention. Dehydrogenation mode is typically carried out for a first time interval and regeneration mode is typically carried out for a second time interval. The durations of the first and second time intervals can be the same, but this is not required. Typically, the duration of the first time interval in at least twice the duration of the second time interval, e.g., at least five times, such as at least ten times the duration of the second time interval. Typically the first and second time intervals do not overlap. Valve means can be utilized for controlling the flows of feeds, reaction product, optional sweep fluids, etc., during the and between the first and second time intervals.

The alkane-containing feed, e.g., the first feed, is provided for catalytic dehydrogenation to produce olefin. Depending, e.g., on the alkane compounds present in the first feed, the process can be carried out to produce particular olefin compounds, such as a desired olefin compound. Typically, when the alkane compound is one having n carbon atoms, with n being an integer ≧2, the alkane compound and the desired olefin compound are of the same order, i.e., have the same value of n. One representative alkane-containing feed comprises ≧50 mole % of propane, e.g., ≧75 mole %, or ≧95 mole %.

In certain aspects, the alkane-containing feed comprises a designated alkane compound selected from among those C_{n|} alkanes of n≧2. For example, the designated alkane compound can be selected from among alkanes having from 2 to 20 carbon atoms, such as from 3 to 12 carbon atoms. The designated alkane compound is one normal alkane, isoalkane, or cycloalkane compound having a single value for n, e.g., n=2, or n=3, or n=4. The designated alkane compound can be, e.g., ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane and 1,3-dimethylcyclohexane. For example, propane can be used as an alkane-containing feed, which can be dehydrogenated to produce propylene, and isobutane can be used as an alkane-containing feed, which can be dehydrogenated to produce isobutylene. In certain aspects, substantially all of the hydrocarbon in the alkane-containing feed is the designated alkane compound, e.g., propylene.

In addition to the designated alkane compound, the alkane-containing feed can further comprise additional hydrocarbon, e.g., additional alkane. When the alkane-containing feed comprises the designated alkane compound and additional hydrocarbon, the alkane-containing feed typically comprises ≧50 wt. % of the designated alkane compound, based on the total weight of hydrocarbon in the alkane-containing feed, e.g., ≧60 wt. %, such as ≧75 wt. %, or ≧90 wt. %, or ≧99 wt. %. Depending, e.g., on the number of carbon atoms in the designated alkane compound, the alkane-containing feed can have a final boiling point of not greater than 200° C., measured according to ASTM D86. Alternatively, the alkane-containing feed stream has a final boiling point of not greater than 150° C., or not greater than 100° C., measured according to ASTM D86.

The alkane-containing feed can be diluted, e.g., with one or more diluents such as one or more substantially-inert materials. For example, the alkane-containing feed stream can be diluted with essentially inert fluid, such as molecular nitrogen. Substantially inert in this context means that ≦0.1 wt. % of the material present in the alkane-containing feed reacts with alkane, molecular hydrogen, and/or olefin under the specified dehydrogenation reaction conditions. The alkane-containing feed typically comprises ≦45 wt. % diluent based on the weight of the alkane-containing feed, e.g., in the range of 1 wt. % to 40 wt. %, such as from 10 wt. % to 30 wt. %.

The alkane-containing feed is preferably substantially free of molecular oxygen. In one aspect, the alkane-containing feed comprises ≦5 mole % molecular oxygen (O₂). Alternatively, the alkane-containing feed that enters in the reaction zone comprises ≦3 mole % of molecular oxygen, or ≦1 mole %. It is believed that providing an alkane-containing feed that is substantially-free of molecular oxygen substantially prevents oxidative coupling reactions that would otherwise consume at least a portion of the feed's alkane.

The oxidant-containing feed, comprises at least one oxidant and optionally diluent. Convenient oxidants are normally gaseous, such as molecular oxygen, ozone, and gases which yield oxygen such as N₂O. Materials that are liquid or solid at ambient conditions may also be used provided that they can be facilely introduced into the flow-through reactor. The oxidant-containing feed stream contains sufficient oxidant for storage with the SOC. For example, in one aspect, the oxidant-containing feed comprises ≧0.1 mole % oxidant, based on total amount of the oxidant-containing feed stream, or ≧0.5 mole %. Alternatively, the oxygen-containing feed ≧5 mole % oxidant, or ≧10 mole % oxidant, or ≧20 mole % oxidant, or ≧25 mole % oxidant, based on total amount of the oxygen-containing feed stream. For example, the amount of oxidant can be in the range of from 0.1 mole % to 99.9% oxidant, such as 0.1 mole % to 25 mole % or 0.1 mole % to 1 mole %. The remainder of the second feed can be diluent, e.g., material that is substantially unreactive (or only mildly so) with oxidant under the conditions utilized for replenishing the SOC's oxygen.

The choice of second feed's oxidant and the amount of oxidant in the second feed can depend in part on the active material selected for the dehydrogenation. As disclosed in the de Graaf reference, cited in the BACKGROUND, certain materials that are catalytically active for alkane dehydrogenation, e.g., platinum, exhibit a loss of dispersion in the active material when exposed to oxidant. Accordingly, when the active material's dehydrogenation component includes platinum and/or one or more other dehydrogenation elements exhibiting a similar loss of dispersion in the presence of oxidant, the second feed typically comprises ≦1 mole % of oxidant, based on total amount of oxidant in the second feed, e.g., ≦1 mole % of molecular oxygen, such as 0.1 mole % to 1 mole % molecular oxygen. Alternatively, in aspects where the active material's dehydrogenation component includes chromium-based material, the second feed can comprise ≧1 mole % of oxidant, e.g., ≧10 mole %, such as ≧15 mole %, or ≧25 mole % . For example the second feed can comprise 1 mole % to 99.9 mole % of molecular oxygen, e.g., 5 mole % to 50 mole %, such as 10 mole % to 35 mole %.

Typically, the second feed's oxidant includes molecular oxygen (O₂). For example, the oxidant can comprise ≧90.0 mol. % of O₂, e.g., ≧99.0 mol. % of O₂, based on the total amount of the oxidant in the oxygen-containing feed stream. The O₂ can be O₂ in air, or O₂ obtained or derived from air, e.g., by separation. Molecular nitrogen obtained or derived from air can be utilized as diluent. In certain aspects, the oxidant includes molecular oxygen in air, and the diluent includes molecular nitrogen in air. For example, the second feed can comprise, consist essentially of, or consist of air.

### Active Material

The active material can be one which has catalytic alkane dehydrogenation activity and optionally capability for storing oxidant and releasing stored oxidant for combusting the dehydrogenation product's molecular hydrogen. For example, the active material can be one having (i) catalytic dehydrogenation functionality or converting alkane (e.g., C₂ to C₅₋ alkane) in the hydrocarbon reactant to olefin having the same number or range of carbon atoms and (ii) selective hydrogen combustion functionality. For example, the active material can be a single component, dual-function catalytic material, i.e., a catalytic material comprising a component having both dehydrogenation functionality and selective hydrogen combustion functionality. More typically, however, the active material is a multi-component catalyst, i.e., one having a first component having alkane dehydrogenation functionality and ii) at least a second component having hydrogen combustion functionality, especially selective hydrogen combustion functionality. The second component can comprise at least one SOC. Optionally, all of the active material is located in the reaction zone.

The invention encompasses various configurations of the active material. As an example, the catalyst material can comprise the first and second components as mixtures or in layers, with the first and second components being arranged within the reaction zone. Alternatively, or in addition, the active material can be a composite which includes a first component which provides catalytic dehydrogenation functionality and a second component which provides oxidant storage and hydrogen combustion functionalities. The composite's second component can be utilized as a substrate for one or more coatings of the first component, for example, or vice versa. Typically, the first and second components are arranged in a manner in which dehydrogenation selectivity toward the desired olefin is optimized, with the second component being arranged relative to the first so that molecular hydrogen combustion (i) depletes the dehydrogenation product's molecular hydrogen during dehydrogenation and (ii) shifts the reaction zone's temperature profile toward isothermal. In certain aspects, release or contact of the stored oxidant with the molecular hydrogen in carrying out the selective hydrogen combustion reaction is regulated to provide sufficient heat for the endothermic dehydrogenation reaction, but not so much heat as to damage the active material or cause undesired side reactions. The relative amount of first and second components in the active material is selected to achieve the specified functions. Typically, the second component, e.g., one or more SOC's, comprises at least 10 wt. % of the active material, based on the weight of the active material. Alternatively, the second component comprises at least 20 wt. %, or at least 30 wt. %, or at least 40 wt. % of the active material.

Examples of suitable dehydrogenation materials and oxidant storage/molecular hydrogen combustion materials will now be described in more detail. The invention is not limited to these materials, and this description is not meant to foreclose other dehydrogenation materials and oxidant storage/molecular hydrogen combustion materials within the broader scope of the invention.

A. Dehydrogenation Functionality of the Active Material

The active material or first component thereof comprises at least one material having alkane dehydrogenation functionality, particularly non-oxidative alkane dehydrogenation functionality. This functionality can be provided by at least one metal selected from Groups 5-14 of the Periodic Table, including such metals in oxide and/or sulfide form. The active material or first component thereof can further comprise at least one additional material utilized as binder, matrix, and/or support. Typically, such additional materials include one or more inorganic oxides or sulfides, especially those that are stable under process conditions specified for dehydrogenation, combustion, and re-oxidation. For example, the additional material can include one or more inorganic oxides of elements in

Groups 13 and 14 of the Periodic Table, silica and/or alumina. The active material or first component thereof can include one or more mixed metal catalysts, meaning that the active material can comprise more than one metal element having non-oxidative alkane dehydrogenation functionality.

The dehydrogenation functionality of the active material or first component thereof can be provided by one or more of (i) Group 5 metals including V, Nb and Ta, with V being preferred, (ii) Group 6 metals including Cr, Mo, W, with Cr and Mo being preferred, and Cr being particularly preferred, (iii) Group 7 metals including Mn and Re, with Mn being preferred, (iv) Group 8 metals including Fe, Ru and Os, with Fe being preferred, (v) Group 9 metals including Co, Rh and Ir, with Co being preferred, (vi) Group 10 metals including Ni, Pd and Pt, with Ni and Pt being preferred, and Pt being particularly preferred, (vii) Group 11 metals including Cu, Ag and Au, with Cu being preferred, (viii) Group 12 metals including Zn and Cd, with Zn being preferred, (ix) Group 13 metals including Al, Ga, In and Tl, with Ga and In being preferred, and (x) Group 14 metals including Ge, Sn and Pb, with Sn being preferred.

Specific examples of active materials having non-oxidative alkane dehydrogenation catalytic activity under the specified dehydrogenation conditions include those listed in the de Graaf reference cited in the BACKGROUND, the contents of which are incorporated herein by reference. The catalytic metal is typically activated before use, e.g., by reducing the catalytic metal from a higher oxidation state to a lower one. Conventional methods can be utilized to do so, but the invention is not limited thereto.

Examples of platinum-based dehydrogenation catalysts include platinum supported on alumina and Pt/Sn supported on alumina. The platinum-based dehydrogenation catalysts can further comprise alkaline promoter. Additional metals such as Mg, Zn and/or Ca can be included in the catalyst. Conventional platinum-containing dehydrogenation catalysts can be used, but the invention is not limited thereto.

Deposits such as coke typically accumulate on the active material during dehydrogenation mode, particularly near active material sites participating in the dehydrogenation reaction. One feature of the invention is that selectivity for coke is less than in conventional catalytic alkane dehydrogenation processes. This allows for decreased regeneration mode duration over that of conventional processes. Shortening the duration of regeneration mode, allows for an increase in the second feed's oxidant content without increasing the amount catalyst dispersion loss beyond that observed in the conventional process. Typically, the second feed's oxidant content (e.g., its molecular oxygen content) is ≧1.0 mole %, such as in the range or 10 mole % to 35 mole %. Doing so provides excess oxidant over that needed for replenishing the active material's oxidant capacity. The excess oxidant increases the rate of coke removed from the active material, so that the time needed for coke removal and the time needed for oxidant replacement converge, becoming, for example, of the same order of magnitude. This desirably shortens the duration of the second time interval, leading to greater olefin yield.

Active materials having dehydrogenation activity derived from platinum, e.g., from platinum atoms, platinum ions, and/or platinum in platinum-containing compounds (collectively, platinum-based materials) are suitable for use in the process. Typically the active material exhibits a decrease in catalytic alkane dehydrogenation activity over the course of dehydrogenation mode operation. It is believed that this activity loss results from at least (i) sensitivity of the active materials' dehydrogenation sites to byproducts produced during dehydrogenation mode, particularly sensitivity to water produced by the molecular hydrogen production, and (ii) accumulation of coke on or proximate to the active material, particularly proximate to the active material's dehydrogenation sites. Compared to active materials having a platinum-based dehydrogenation functionality, active materials having a chromium-based dehydrogenation functionality have been found to be less sensitive to water without exhibiting an appreciably greater deactivation rate from coke accumulation during dehydrogenation mode. In certain aspects, the active material or the first component thereof comprises at least one chromium-based material, optionally in combination with at least one platinum-based material.

Besides being more resistant to water deactivation than platinum-containing active materials, chromium-containing active materials are also less susceptive to deactivation from coking during dehydrogenation/oxidation reactions during dehydrogenation mode and less susceptive to dispersion loss during regeneration mode. The chromium-based dehydrogenation catalysts are also less susceptible to destruction resulting from high temperature spikes, which may arise at times during regeneration mode. Examples of preferred chromium-containing active materials include chromia supported on alumina, such as those described in J. Gascon, et al., Propane Dehydrogenation over a Cr₂O₃/Al₂O₃ Catalyst: Transient Kinetic Modeling of Propane and Coke Formation, Applied Catalysis A: General 248 (2003) 105-116, which is incorporated by reference herein in its entirety. The chromium-containing active materials can further comprise alkaline promoter.

B. Hydrogen Combustion-Oxidant Storage Functionality of the Active Material

Besides dehydrogenation activity, the active material or second component thereof optionally has selective hydrogen combustion and oxidant storage functionality. For example, the active material can comprise at least one SOC having selective hydrogen combustion functionality and oxidant storage functionality. The SOC can comprise a metal oxide, for example a transition metal oxide, having a reversible sorptive affinity for oxidant at elevated temperature. In this context, the term “elevated temperature” means a temperature in the range of from 400° C. to 1000° C., and the term “high sorptive capacity” means an oxygen storage capacity of at least 40 millimoles of oxygen per mole of the SOC that contacts the oxygen at a temperature of 800° C. Such materials include those that sorptively remove and release oxidant and those that undergo a chemical and/or physical change in the course of reversible oxidant storage. The SOC can be one that stores oxidant in molecular form, e.g., as molecular oxygen, but this is not required. In certain aspects, the SOC has capacity for storing and releasing oxidant in atomic or ionic form, e.g., as oxygen atoms and/or oxygen ions. In certain aspects, the SOC can enable the bulk separation and purification of oxygen based on ionic transport, in which the SOC is maintained at high temperature to temporarily store oxygen. Oxygen that contacts the surface of the SOC can be decomposed on the surface of the material and incorporated into the crystalline lattice of the material. Storage of the oxygen can be particularly facilitated over the temperature range from 400° C. to 1000° C.

In certain aspects, when oxidant contacts the SOC, oxidant (typically molecular oxygen, but not limited thereto) is adsorbed and dissociated, with charge transfer acting to cause penetrative flux of oxidant into the SOC. A chemical potential driving force can be employed to effect ionic transport of oxidant into the SOC.

The SOC can be of any suitable size, shape and conformation appropriate to oxidant storage and molecular hydrogen combustion. For example, the material can be in a finely divided form, e.g., beads, spheres, rings, toroidal shapes, irregular shapes, rods, cylinders, flakes, films, cubes, polygonal geometric shapes, sheets, fibers, coils, helices, meshes, sintered porous masses, granules, pellets, tablets, powders, particulates, extrudates, cloth or web form materials, honeycomb matrix monolith, composites (of the SOC with hydrocarbon conversion catalyst and/or support material), including in comminuted or crushed forms. The SOC can be mixed with or coated onto a support or substrate. For example, the SOC can be in the form of finely-divided materials as a part of a thermal support or as one or more coatings on a thermal support substrate to provide a material having oxygen-storage functionality. For example, the SOC can be included as a coating onto, mixture with, or otherwise associated with a substantially inert substrate included in or with the active material.

In certain aspects, the SOC can be formed by metal-organic chemical vapor deposition (MOCVD) on suitable supports or substrates using appropriate precursors for the respective metal components of the SOC. Use of MOCVD allows relatively close control of stoichiometry and uniformity of coverage to be achieved. MOCVD can be used to deposit films of multicomponent SOCs with compositional reproducibility on the order of 0.1% and thickness uniformity of better than 5%.

Alternatively, the SOC can be formed as bulk articles, e.g., particles, by various manufacturing techniques. Such techniques include powder metallurgy, slurry metallurgy (slip casting, tape casting, etc.) and coextrusion.

Another alternative technique for forming the SOC can be a sol gel technique. Such technique can be advantageous when the SOC is deposited on an inert substrate comprising porous silica, alumina, kieselguhr, or the like. Sol gel techniques can be employed to make up a sol of the precursor constituents of the SOC and to spray, dip-coat, soak, roller coat, or otherwise apply the solution to the substrate. The coated substrate containing the precursor material can be subjected to high temperature, e.g., calcined, to produce the desired SOC.

Transition metal oxides are particularly useful as SOCs. Transition metals can be considered an IUPAC Group 3-12 element and elements of the Lanthanide series. In one aspect, the SOCs comprise oxides containing at least one Group 3, Group 6, Group 7, Group 8, Group 9 and Lanthanide series element of the Periodic Table. In other aspects, the SOCs comprise at least one metal-based component comprised of one or more elements from Groups 1, 2 and 3; one or more elements from Groups 4-15; and at least one of oxygen and sulfur. Perovskites and related materials, such as perovskite-like materials and pyrochlores, can be particularly useful as SOCs. “Perovskites” are typically oxygen-containing compounds having the crystal structure, ABO₃, with high-temperature O²⁻ vacancies. Such structures can also be denoted by use of the symbol δ, according to the general formula ABO_{3-δ}. The “A”-site cations can be rare earth (e.g., Lanthanide series including La and Y), alkaline earth (i.e., Group 2), alkaline (Group 1) and large cations such as Pb²⁺, Bi³⁺, or Ce⁴⁺. The “B”-site cations can be 3d, 4d, or 5d transition-metal cations. Multiple cation-type occupations are possible. Framework sites “A” and “B” can be dodecahedral and octahedral, respectively, cf., L. G. Tejuca and J. L. Fierro, Properties and Applications of Perovskite-type Oxides, Marcel Dekker, New York, 1993.

Conventional perovskite remains stable and reversible with regard to changes of δ within a certain range: The value δ can be up to 0.25; for example δ can be from 0.05 to 0.25 (although higher values have been reported), at elevated temperature and low oxygen partial pressure, i.e., δ is a function of temperature and partial pressure of oxygen. Perovskite stability can be governed by cation radii of lattice metals in various valence states combined into a parameter “t” called “tolerance factor”, cf. , Z. Shao, et al., Sep. Purif Technol., 25 (2001) 419-42. A perovskite structure can be formed at t ranges from 0.75-1.

Examples of suitable perovskite are disclosed in U.S. Pat. No. 7,338,549. Typically, the perovskite has the general formulas (1), (2), and (3):

AₓB_{y}O_{3-δ} (1)

AₓA′^{x′}B_{y}B′^{y′}O_{3-δ} (2)

AₓA′_{x′}A″_{x″}B_{y}B′_{y′}B″_{y″}O_{3-δ} (3)

and combinations thereof. In these equations, A, A′, and A″ are independently selected from ions of atoms having atomic number ranging from 57-71, inclusive, a cation of yttrium, ions of Group 1 atoms, ions of Group 2 atoms, and combinations of two or more, where Group 1 and Group 2 refer to the periodic table of elements. B, B′, and B″ are independently selected from d-block transition-metal ions selected from Mn, Cr, Fe, Co, Ni, and Cu. The values of x, x′, x″, y, y′, and y″ are each real numbers ranging from 0 to 1.0, and x+x′+x″=0.8-1.0; y+y′+y″=1.0; and δ ranges from about 0.05 to about 0.30.

Compounds isostructural with perovskite (“perovskite-like compounds”) are also suitable SOCs, including those having general formulas (4), (5), (6), (7), (8), and (9):

A₂BO_{4-δ} (4)

A₂B₂O_{5-δ} (5)

AO(ABO_{3-δ})ₙ (6)

AM₂Cu₃O_{7-δ} (7)

Bi₄V₂₍₁₋ₓ₎Me₂ₓO₁₁₋₃ₓ (8)

A″B″O₃ (9)

In these equations, A is independently selected from ions of atoms having atomic numbers ranging from 57-71, inclusive, a cation of yttrium, ions of Group 1 atoms, ions of Group 2 atoms, and combinations of two or more, where Group 1 and Group 2 refer to the periodic table of elements. B is independently selected from d-block transition metal ions. A″ is an ion of Na or Li, and B″ is an ion of W or Mo. M is a metal cation selected from cations of Group 2 atoms of the periodic table of elements. Me is a metal cation selected from cations of Cu, Bi, and Co atoms. The value for x is a real number ranging from 0.01 to 1.0, n is in the range of from 1 to about 10; and δ ranges from about 0.05 to about 0.30.

Pyrochlores are also suitable SOCs, including those Pyrochlores having the general formula (10):

A₂B₂O_{7-δ} (10)

In this equation, A is independently selected from ions of atoms having atomic numbers ranging from 57-71, inclusive, a cation of yttrium, ions of Group 1 atoms, ions of Group 2 atoms, and combinations of two or more, where Group 1 and Group 2 refer to the periodic table of elements. B is independently selected from d-block transition metal ions; and δ ranges from about 0.05 to about 0.30.

SOCs can also be selected from among cerium-containing and praseodymium-containing metal oxide, including one or more of CeO₂, Pr₆O₁₁, CeO₂—ZrO₂, CuO—CeO₂, FeOₓ—CeO₂ (1.0≦X≦1.5), MnOₓ—CeO₂ (1.0≦X≦3.5), and Pr₆O₁₁—CeO₂.

Certain suitable SOCs include at least one metal-based component comprising one or more elements from Groups 1, 2 and 3 of the Periodic Table; one or more elements from Groups 4-15; and at least one of oxygen and sulfur. Examples of these SOCs are described in U.S. Pat. Nos. 7,122,492; 7,122,493; 7,122,495; and 7,125, 817, the descriptions of each being incorporated herein by reference.

The arrangement and distribution of the dehydrogenation functionality and the oxidant storage-molecular hydrogen combustion functionality in the active material is not critical, although it is beneficial for these functionalities to be located proximate to one another, e.g., as an active material composite. Suitable active material composites will now be described in more detail.

C. Active Material Composites

In certain aspects, the active material is a composite of a first component having dehydrogenation functionality and at least a second component, the second component having oxidant storage-molecular hydrogen combustion functionality. The composite optionally includes additional material, such as support, binder, or matrix material. The composite can be, e.g., a mixed metal oxide comprising: i) a first component comprising metal oxide having catalytic alkane dehydrogenation functionality, and ii) a second component comprising metal oxide having selective hydrogen combustion functionality and oxygen-storage functionality. The first component can be arranged as dehydrogenation catalyst particles in a physical mixture with a support material. Alternatively, the dehydrogenation catalyst particles can be deposited on the support material. The second component can also be included with the physical mixture of first component and support material; alternatively in a mixture with a support material without the first component; alternatively deposited on a support material. Optionally, the second component's support material is the same as the first component's support material.

The composite can include one or more of several forms. For example, the composite can include at least one staged composite. A “staged composite” is one in which the first component's metal oxide is deposited on or in a substrate upstream of the second component's metal oxide. Alternatively, or in addition, the composite can include at least one layered composite. A “layered composite” is one in which the metal oxide having alkane dehydrogenation functionality can be arranged as a surface layer, with the metal oxide having selective hydrogen combustion functionality and oxygen-storage functionality being arranged as a sub-surface layer. Alternatively or in addition, the composite can include at least one monolith. Typical monoliths include ceramic structures having a plurality of substantially parallel internal passages, e.g., those in the form of a ceramic honeycomb. The first and second components can be included in the monolith, or as one or more coatings of first and/or second components on the monolith's inner surfaces, such as on the internal surfaces of one or more of the passages.

The active material composite's first and second components are typically incorporated within the reaction zone of the reactor to conveniently establish flow of the first and second feed through a flow-through reactor such as a tube reactor. The second component's SOC is preferably arranged in the reaction zone of the reactor to release oxidant from the SOC as the dehydrogenation product's molecular hydrogen flows over or across the SOC in the reaction zone. Monolithic active material composites are particularly desirable when the reaction is carried out in one or more tube reactors, the monolith being a convenient form for supplying feeds to the reaction zone, conducting product away from the reaction zone, and adding or removing heat (e.g., via a flow of heat transfer fluid within at least a portion of the monolith's internal passages), in order to establish and maintain a substantially isothermal temperature profile in the reaction zone.

### Reactor and Process Conditions

Examples of reactors and process conditions that are useful in certain aspects of the invention will now be described in more detail. The invention is not limited to these reactors and process conditions, and this description is not meant to foreclose other reactors and/or process conditions within the broader scope of the invention.

The reaction zone is typically located in a reactor, such as a tube reactor. A plurality of reactors can be used, e.g., a plurality of reactors arranged in series, parallel, or series-parallel. Typically, the reaction zone is substantially isothermal at the start of the process, but this is not required since isothermal conditions can be established during the course of the dehydrogenation reaction in dehydrogenation mode. Dehydrogenation mode typically operates for a first time interval, during which a flow of a first feed, an alkane-containing feed, is established into the reaction zone. At least a portion of the first feed's alkane is dehydrogenated in the presence of a catalytically effective amount of at least one active material having first and second components. The first component, which comprises e.g., at least one chromium-containing dehydrogenation catalyst, is present in a sufficient amount to provide catalytic alkane dehydrogenation functionality under the specified process conditions. The second component, comprising e.g., at least one SOC alkane, is present in a sufficient amount to provide oxidant storage and selective hydrogen combustion functionality under the specified conditions. During dehydrogenation mode, the reaction zone is maintained under isothermal conditions, typically by adding or removing heat from reaction zone components, feed or components thereof, and/or reaction product or components thereof. At least a portion of the molecular hydrogen in the dehydrogenation product is combusted in the reaction zone in the presence of an oxidant that is associated with (e.g., stored in or stored on) the SOC. Combustion of the molecular hydrogen with the stored oxidant produces water in the reaction product, which can be separated from the reaction product, e.g., downstream of the reaction zone, such as by one or more of fractionation, extraction, gravitational settling, etc. During dehydrogenation mode, the SOC will be reduced from an oxidized state (SOₓC) to a reduced state SO_{y}C, where x and y are positive real numbers and x>y. Dehydrogenation mode is typically carried out until (i) alkane conversion (indicated by an increase in unreacted alkane in the reaction product) is ≦90% of that at the start of dehydrogenation mode, e.g., ≦75%, or ≦50%; and/or (ii) selectivity for the desired olefin (indicated by the amount of desired olefin in the reaction product) is ≦90% of that at the start of dehydrogenation mode, e.g., ≦75%, or ≦50%. Typically, when this occurs, the flow of the alkane-containing feed stream through the reaction zone is curtailed or ceased, so that regeneration mode can be carried out. In certain aspects, the first time interval is ≧1 second, e.g., ≧100 seconds, such as ≧10⁴ seconds, or even ≧10⁶ seconds.

Regeneration mode typically includes replenishing at least a portion of the SOC's oxidant that was consumed during dehydrogenation mode and removing at least a portion of the active material's accumulated coke. Regeneration mode is typically carried out during a second time interval. The SOC is oxidized (or re-oxidized) during regeneration mode from state SO_{y}C to state SO_{z}C, where z is a positive real number and z>y. Once oxidized to state SO_{z}C, the SOC can again function as a source of oxidant for the selective molecular hydrogen combustion reaction, which is also carried out in the reaction zone. Although z can have substantially the same value as x, this is not required.

In an alternative aspect, regeneration mode is not carried out. In such cases, spent active material or the SOC component thereof is removed from the reaction zone and replaced with fresh or regenerated material having sufficient oxidant content for the dehydrogenation/combustion. Replacement can be carried out continuously, e.g., utilizing conventional fluidized catalyst or slurry catalyst technology, in a batch method, and in combinations thereof.

The reaction zone can be substantially isothermal during the second time interval, but this is not required. At least a portion of the second feed's oxidant, typically oxygen, is stored by the active materials' oxidant storage sites. The second feed's oxidant is also used for removing coke deposits from the active material, particularly proximate to the active material's dehydrogenation sites, which restores the active material's dehydrogenation activity. Once sufficient re-oxidation has occurred, i.e., sufficient oxidant is stored to carry out the molecular hydrogen combustion during dehydrogenation mode, the flow of the second feed through the reaction zone can be curtailed or ceased, and the flow of first feed re-established.

Since selectivity for coke is less than in conventional catalytic alkane dehydrogenation processes, the duration of regeneration mode is less than that of conventional processes, particularly when using active materials which include Chromium as a dehydrogenation component. Shortening the duration of regeneration mode has the desirable feature that the second feed's oxidant content can be increased without increasing the amount catalyst dispersion loss beyond that observed in the conventional process. Typically, the second feed's oxidant content (e.g., its molecular oxygen content) is ≧1.0 mole %, such as in the range or 10 mole % to 35 mole %. Doing so provides excess oxidant over that needed for replenishing the active material's oxidant capacity. The excess oxidant increases the rate of coke removed from the active material, so that the time needed for coke removal and the time needed for oxidant replacement converge, becoming, for example, of the same order of magnitude. This desirably shortens the duration of the second time interval, leading to greater olefin yield. Typically, regeneration mode is carried out for a sufficient time to (i) replenish ≧50% (wt. basis) of the SOC's original oxidant storage capacity, e.g., ≧75%, such as ≧90%, and /or (ii) remove ≧50% (wt. basis) of accumulated coke on the active material, e.g., e.g., ≧75%, such as ≧90%. Generally, the duration of regeneration mode is ≦50% of the duration of dehydrogenation mode, e.g., ≦25%, such as ≦10%. In certain aspects, the duration of regeneration mode is ≦5×10⁴ seconds, e.g., ≦1×10³ seconds, such as ≦100 seconds, or ≦10 seconds.

Alternating flows of first and second feeds, e.g., alternating first and second time intervals can be repeated continuously or semi-continuously. One or more additional feeds, e.g., one or more sweep fluids, can be utilized between flows of the first and second feeds, e.g., to remove undesired material from the reactors, such as non-combustible particulates including soot. The additional feeds are typically inert under conditions specified for the first and second time intervals.

The reaction zone can include one or more fixed bed reactors containing the same or different catalysts, a moving bed reactor, or a fluidized bed reactor. The feeds may be contacted with the active material in one or more of an upward, downward, or radial flow fashion. The feeds and reaction product removed from the reaction zone may be in the liquid phase, mixed liquid and vapor phase, or typically in the vapor phase. Typically a fixed bed reactor is employed, e.g., one having a plurality of beds of one or more of the specified active materials. When the reaction zone contains a plurality of beds of active material, the active material of each bed can be of the same composition (e.g., using the same type and amount of dehydrogenation component and SOC), but this is not required.

During dehydrogenation mode, the reaction zone is maintained or controlled to have a substantially isothermal temperature profile at about the reaction zone's average temperature. In one aspect the reaction zone's average temperature is in a range of from 400° C. to 700° C. Alternatively, the reaction zone's average temperature is in a range of from 450° C. to 650° C., or in a range of from 400° C. to 600° C. In another alternative embodiment, the reaction zone's average temperature is in a range of from 400° C. to 500° C. It is observed that a lower average temperature in the reaction zone, e.g., in the range of from 400° C. to 500° C., increases yield of the desired olefin and decreases the yield of undesired by products such as methane. The reaction zone can be controlled, e.g., to exhibit a temperature profile having a variance from the average temperature of ≦+/−10° C. during dehydrogenation mode, e.g., ≦+/−5° C. Appropriate thermal regulation of the selective hydrogen combustion reaction can be carried out through the use of one or more SOCs, and by distributing the SOC in the reaction zone to achieve a substantially isothermal profile. Additional regulation of the heat of the reaction, if needed, can be carried out through thermal moderation of the temperature profile in the reaction zone. Typically, establishing and maintaining an isothermal temperature profile is carried out by (i) selecting an appropriate type and amount of SOC, (ii) appropriate distribution of the SOC in the active material, and (iii) additional thermal moderation of the reaction zone to achieve a more isothermal temperature profile.

The type and amount of SOC are selected to provide sufficient desorbed oxidant for molecular hydrogen combustion under the specified dehydrogenation conditions, but little if any additional oxidant beyond what is needed for the combustion. Since adsorption is typically exothermic and desorption typically endothermic, desorption of additional oxidant would undesirably compete with the endothermic dehydrogenation reaction for heat produced by the combustion, resulting in a deviation of the reaction zone from an isothermal temperature profile. Consequently, it is also desirable for the SOC to be one having a heat of desorption that is less than the heat needed for the dehydrogenation. For at least these reasons, SOCs comprising perovskite, material isostructural with perovskite, pyrochlore, and/or material isostructural with pyrochlore are particularly suitable, especially such of those having a pore size appropriate for selective molecular hydrogen combustion. Since an isothermal temperature in the reaction zone is easier to maintain when desorbed oxidant combustion occurs proximate to the active material's dehydrogenation sites, it is desirable for the active material to include at least one composite, the composite including dehydrogenation component impregnated into and/or onto at least one SOC component of suitable porosity for selective molecular hydrogen combustion. Typically, the impregnation is of substantially-uniform concentration over the SOC. Suitable active materials include SOCs comprising perovskite, material isostructural with perovskite, pyrochlore, and/or material isostructural with pyrochlore, the SOC being one of suitable porosity and impregnated with ≧0.5 wt. % of at least one dehydrogenation metal such as Chromium.

Additional thermal moderation of the reaction zone's temperature profile, if needed, can be carried out by removing heat from or adding heat to one or more locations in the reaction zone. Typical methods for doing so include external or internal transfers of heat to and/or from one or more beds of the active material. Examples of reactors configured to do this include radial flow catalyst bed reactors having heat transfer tubes arranged within the beds. The heat transfer tubes can be arranged in a variety of configurations including vertical, horizontal, and/or helical tubular arrangements heat modulation to achieve substantially isothermal reaction zone conditions. In certain aspects, the reaction zone includes at least one bed of active material and a plurality of helical tubes within bed. During dehydrogenation mode, a heat transfer fluid is conveyed through the tubes, the temperature of the heat transfer fluid being regulated to supply or remove heat in order to maintain the bed in a substantially isothermal profile. The catalyst bed can be arranged axially and/or radially within the reaction zone, in proximity to the heat transfer tubes. Such reactor zones are described German Patent Application No. DE-A-3 318 098, and in U.S. Pat. No. 4,339,413 and U.S. Pat. No. 4,636,365, which are incorporated by reference herein in their entirety. Preferably, at least 50% of the heat transfer tubes in the reaction zone are configured so that they are each exposed to substantially the same thermal load during dehydrogenation mode. For example, when additional heat is removed from the reaction zone by a heat transfer fluid comprising liquid water, at least 50% of the tubes produce the same amount of steam (uniform distribution of the water and steam inside the tubes). Such reaction zones are disclosed in U.S. Pat. No. 6,958,135, which is incorporated by reference herein in its entirety. Typically ≧25 mole % of the dehydrogenation product's molecular hydrogen is consumed during dehydrogenation mode, e.g., ≧50 mole %, such as ≧75 mole %, or ≧90 mole %. It is also typical that ≧25 mole % of molecular hydrogen combustion occurring during dehydrogenation mode be carried out using oxygen released from at least one SOC, e.g., ≧50 mole %, such as ≧75 mole %, or ≧90 mole %.

Alternatively or in addition to the use of heat transfer tubes, the first feed can be heated before or during its introduction into the reaction zone to provide additional thermal moderation. Conventional heating means can be utilized to do this, but the invention is not limited thereto. The preheating can be applied throughout dehydrogenation mode, or more typically, for an initial period at the start of the first interval in order to initiate the dehydrogenation reaction. After this initial period, heat released in the reaction zone by combustion of the dehydrogenation product's molecular hydrogen can substitute for at least a portion of the first feed preheating for maintaining the established isothermal temperature profile for the remainder of dehydrogenation mode.

During the dehydrogenation mode, the reaction zone is maintained or controlled at a pressure effective for carrying out the specified dehydrogenation and molecular hydrogen combustion reactions. In certain aspects, the reaction zone pressure is ≧0 psia (0 kPa absolute) and ≦500 psia (3447 kPa absolute). Alternatively, the reaction zone pressure is in a range of from 5 psia (34 kPa absolute) to 200 psia (1379 kPa absolute) or in a range of from 10 psia (69 kPa absolute) to 100 psia (689 kPa absolute) or in a range of from 15 psia (103 kPa absolute) to 50 psia (344 kPa absolute).

The flow of alkane-containing feed into the reaction zone is carried out to achieve a weight hourly space velocity (WHSV) effective for carrying out the catalytic dehydrogenation process. In one embodiment, the WHSV is from 0.1 hr⁻¹ to 100 hr⁻¹. Alternatively, the WHSV is from 0.5 hr⁻¹ to 50 hr¹. The catalytic dehydrogenation reaction process of this invention provides superior overall dehydrogenation properties including one or more of high selectivity to desired olefin, high conversion of alkane and low active material deactivation rate compared to conventional processes. Selectivity to desired olefin can be, e.g., at least 40%, or at least 50%, or at least 60%, or at least 80% on a molar basis. In particular, when the designated alkane is propane, the process has high selectivity to propylene. As another example, when the designated alkane is isobutane, the process has high selectivity to isobutene. Alkane conversion, (e.g., Propane conversion) is typically at least 50% on a molar basis, or at least 60%, or at least 80%, or at least 90%.

Olefin compounds produced by the process can be cyclic or acyclic, meaning that olefin's double bond can be located between carbon atoms forming part of a cyclic (closed-ring) or part of an open-chain grouping, although typically the olefin is acyclic. The olefin can have more than one double bond, although typically the olefin has one double bond only. The process is particularly effective in converting a lower alkane, e.g., a particular C₃-C₅ alkane, to respective lower olefin of the same carbon number, e.g., a particular C₃-C₅ olefin.

### Recovery and Use of the Reaction Product's Olefin

The reaction product typically comprises at least one desired olefin, water, unreacted alkane, other unreacted first feed components, unreacted molecular hydrogen, etc. When present, the amount of unreacted alkane is small, typically ≦10% (mole basis), e.g., ≦1%. When present, the amount of unreacted molecular hydrogen is also small, typically ≦1% (mole basis), e.g., ≦0.1%. Olefin can be removed from the reaction product by any convenient method, e.g., by cone or more conventional methods. One such method includes cooling the reaction product to condense the reaction product's water and any heavy hydrocarbon that may be present, leaving the olefin and any unreacted alkane primarily in the vapor phase. Olefin and unreacted alkane can then be removed from the reaction product in one or more separator drums. Olefin can be separated from unreacted alkane in one or more splitters, e.g., at least one splitter can be used for separating propylene produced in the process from a first feed comprising propane. The greater alkane conversion, greater olefin selectivity, and the more efficient consumption within the reaction zone of at least a portion (e.g., substantially all) of the dehydrogenation product's molecular hydrogen results in a considerable simplification in reaction product separation requirements over the conventional process. As a result, the invention is more efficient and has lesser energy requirements relative to conventional alkane dehydrogenation processes.

In particular aspects, the recovered olefin can be used for producing polymer, e.g., recovered propylene can be polymerized to produce polymer having segments derived from the recovered propylene such as polypropylene, ethylene-propylene copolymer, etc. Recovered isobutene can be used, e.g., for producing one or more of oxygenate like MTBE, fuel additives like diisobutene, synthetic elastomeric polymer like butyl rubber, etc.

### EXAMPLES

A process for non-oxidative catalytic dehydrogenation of propane in accordance with the invention is simulated. The simulation is carried out using a gPROMS program (gPROMS, version 2.1.1, PSE Enterprise Ltd., London, England). A comparative simulation is also carried out, but in a reaction zone that does not have an isothermal temperature profile. In the comparative simulation, an adiabatic packed bed reactor (no heat removal from the reaction zone) is used instead of a substantially isothermal reaction zone.

In both simulations, the active material comprises i) a first component having PDH functionality and ii) a second component comprising SOC having SHC functionality. The active material comprises a 50/50 mixture of the first and second components.

At the start of the simulation and comparative simulation, the reactor is flushed with molecular nitrogen and heated to an average reaction zone temperature of 550° C., a temperature at which propane dehydrogenation occurs immediately. A flow of first feed comprising 100% propane is established into the reaction zone at a WHSV of 1 hr⁻¹. The dehydrogenation reaction in the reaction zone converts a portion of the feed's propane to produce a dehydrogenation product comprising propylene and molecular hydrogen. The molecular hydrogen reacts with oxygen released from the SHC—SOC component and combusts to produce water. In both the simulation and comparative simulation, the rate of combustion is carried out so that the propane dehydrogenation equilibrium reaction is not reached, i.e., equilibrium is shifted toward production of the olefin and away from the reverse reaction which converts molecular hydrogen and olefin back to propane.

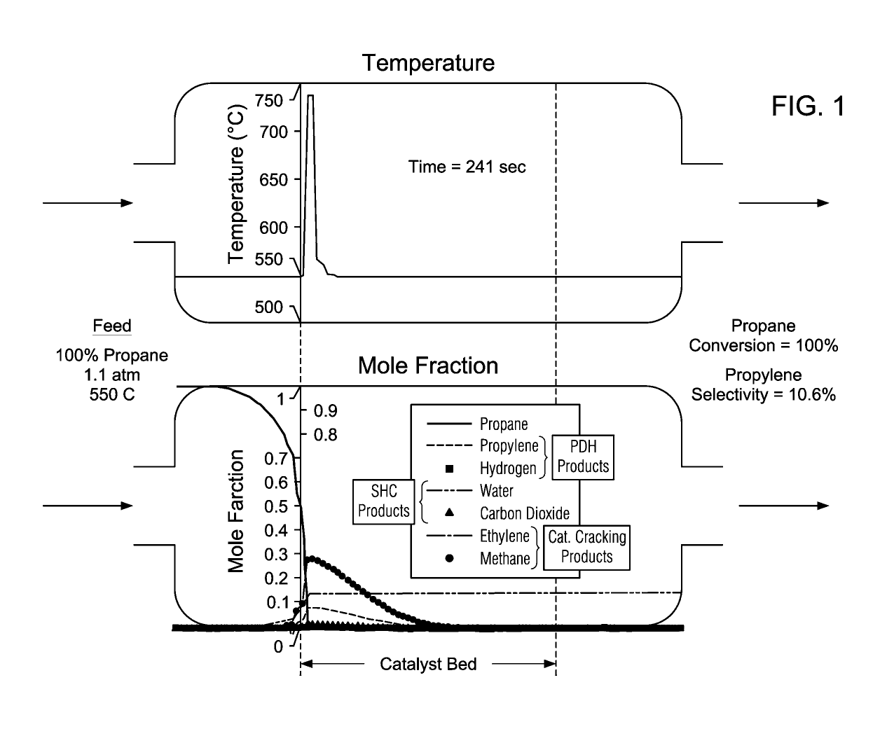

In the comparative simulation, illustrated schematically in FIG. 1, the process is carried out at adiabatic conditions and the reaction zone is not substantially isothermal. In other words, heat is neither added nor removed from the reaction zone during the dehydrogenation, resulting in a non-isothermal temperature profile exhibiting a temperature at certain locations within the reaction zone rising from 550° C. at the start of the simulation to well above 650° C. At a temperature of 650° C., the process is non-selective for propylene production via propane dehydrogenation; undesired cracking reactions predominate instead. As FIG. 1 shows, the primary products of the comparative example are methane and ethylene. Although not shown in the model, it can be expected that the comparative simulation also leads to severe catalyst deactivation from coke formation and sintering of the active material as the reaction is continued.

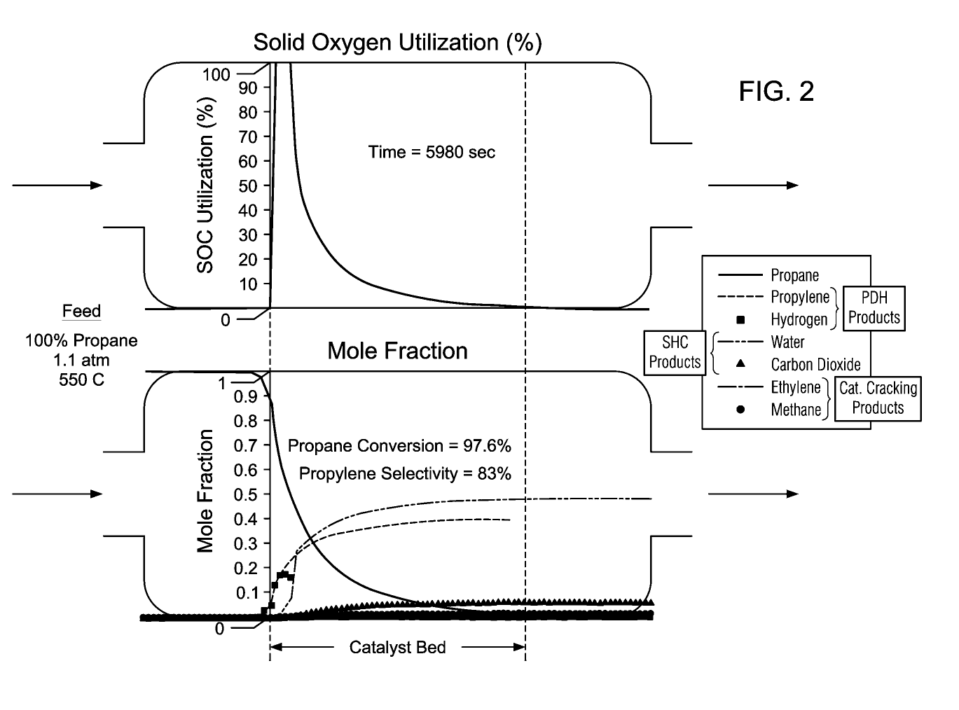

The simulation carried out in accordance with the invention is illustrated schematically in FIG. 2. In this simulation, the reaction zone is maintained in a substantially isothermal profile at about the initial average reaction zone temperature of about 550° C. By maintaining the essentially isothermal temperature profile around the desired average temperature, substantially all of the dehydrogenation product's molecular hydrogen is consumed, with propane conversion being 97.6% and selectivity to propylene being 83%. This is a substantial improvement over the comparative example, which showed a propane conversion of 100%, but at a very low selectivity to propylene at 10.6%. Such an improvement could not have been reasonably expected given very low selectivity to olefin yielded from the comparative example.

While the invention has been described and illustrated with respect to certain aspects, it is to be understood that the invention is not limited to the particulars disclosed and extends to all equivalents within the scope of the claims. Unless otherwise stated, all percentages, parts, ratios, etc. are by weight. Unless otherwise stated, a reference to a compound or component includes the compound or component by itself as well as in combination with other elements, compounds, or components, such as mixtures of compounds. Further, when an amount, concentration, or other value or parameter is given as a list of upper preferable values and lower preferable values, this is to be understood as specifically disclosing all ranges formed from any pair of an upper preferred value and a lower preferred value, regardless of whether ranges are separately disclosed. All patents, test procedures, and other documents cited herein, including priority documents, are fully incorporated by reference to the extent such disclosure is not inconsistent and for all jurisdictions in which such incorporation is permitted.

1. A process for converting alkane to olefin, comprising:
(a) providing an alkane-containing feed comprising at least one C₂₊ alkane;
(b) providing a substantially isothermal reaction zone having an average temperature in the range of from 400° C. to 700° C.;
(c) providing at least one active material located in the reaction zone, the active material having catalytic alkane dehydrogenation functionality at the average temperature;
(d) (i) establishing a flow of the alkane-containing feed into the reaction zone, and dehydrogenating at least a portion of the alkane in the flow of alkane-containing feed to produce a dehydrogenation product comprising olefin and molecular hydrogen, the dehydrogenating being carried out in the reaction zone in the presence of at least a portion of the active material,
(ii) combusting at least a portion of the dehydrogenation product's molecular hydrogen in the reaction zone during the catalytic dehydrogenation to produce a combustion product comprising water; and
(iii) maintaining the reaction zone substantially isothermal at the average temperature during the dehydrogenating and combusting; and
(e) conducting away from the reaction zone a reaction product comprising at least a portion of the dehydrogenation product's olefin and at least a portion of the combustion product's water.

2. The process of claim 1, wherein the substantially isothermal reaction zone has a temperature profile exhibiting a variance from the average temperature of not greater than +/−10° C. during step (d).

3. The process of claim 1, wherein the substantially isothermal reaction zone has a temperature profile exhibiting a variance of not greater than +/−5° C. during step (d).

4. The process of claim 1, wherein the reaction zone is maintained substantially isothermal during step (d) by transferring heat away from the reaction zone.

5. The process of claim 1, wherein ≧60 mole % of the alkane-containing feed's C₂₊ alkane is dehydrogenated during step (d).

6. The process of claim 1, wherein ≧25 mole % of the dehydrogenation product's molecular hydrogen is combusted during step (d).

7. The process of claim 1, wherein the average temperature is in the range of 450° C. to 550° C., and the dehydrogenating and combusting are carried out at a pressure in the range of from 0.1 bar to 10 bar and an alkane-containing feed space velocity WHSV ≧0.5 hr⁻¹.

8. The process of claim 1, wherein the active material does not include oxidative alkane dehydrogenation functionality.

9. The process of claim 1, wherein (i) the active material comprises first and second components, the first component having the catalytic alkane dehydrogenation functionality;
(ii) the second component has functionality for selectively combusting the molecular hydrogen in step (d); (iii) the combustion is selective for the molecular hydrogen combustion;
(iii) the second component has functionality for storing oxygen and for releasing oxygen during the selective combustion; (iv) the selective combustion is carried out proximate to the dehydrogenation.

10. The process of claim 9, wherein the first component comprises ≧1 wt. % of at least one dehydrogenation element selected from Groups 5-13 of the Periodic Table and the second component comprises ≧1 wt. % of at least one solid oxygen carrier, wherein at least a portion of the second component's selective hydrogen combustion functionality and at least a portion of the second component's oxygen storage functionality are derived from the solid oxygen carrier.

11. The process of claim 10, wherein the dehydrogenation element is platinum and/or chromium and the solid oxygen carrier comprises oxide of (i) one or more transition metals selected from Groups 3-13 of the Periodic Table and/or (ii) one or more lanthanides.

12. The process of claim 11, wherein (i) the dehydrogenation element is chromium, (ii) the first component further comprises alumina, and (iii) the solid oxygen carrier is one or more of perovskite, pyrochlore, material isostructural with perovskite, and material isostructural with pyrochlore.

13. The process of claim 9, wherein the active material is a mixture of the first and second components, the mixture being in the form of a fixed bed in the reaction zone.

14. The process of claim 9, wherein the active material is a catalytic composite of the first and second components, the composite being located within the reaction zone.

15. The process of claim 10, wherein the molecular hydrogen is selectively combusted through contact with oxygen stored in the second catalyst component.

16. The process of claim 10, wherein the solid oxygen carrier is reduced from a first state SOₓC to a second state SO_{y}C during step (d), wherein x is a positive real number ≧0 and y is a positive real number that is <x, and further comprising:
(f) curtailing or substantially halting the flow of the alkane-containing feed;
(g) establishing a flow of an oxidant-containing feed into the reaction zone;
(h) reacting the solid oxygen carrier with the oxidant-containing feed to oxidize the solid oxygen carrier from the second state to a third state SO_{z}C, where z is a positive real number >y;
(i) curtailing or substantially halting the flow of oxidant-containing feed; and
(j) repeating steps (d) and (e).

17. The process of claim 16, wherein (i) the dehydrogenation element is chromium, (ii) the first component further comprises alumina, (iii) the solid oxygen carrier is one or more of perovskite, a compound isostructural with perovskite, and pyrochlore, (iv) the alkane-containing feed comprises ≧90 mole % propane, and (v) the reaction product's olefin comprises ≧90 mole % propylene.

18. The process of claim 17, wherein ≧50 mole % of the propane in the alkane-containing feed is converted in step (d) to propylene in the reaction product.

19. The process of claim 17, wherein the alkane-containing feed is substantially free of molecular oxygen.

20. The process of claim 17, wherein the oxidant-containing feed comprises molecular oxygen in air.

21. A heat-transfer, catalytic alkane dehydrogenation reaction process for producing an olefin-rich product, the process comprising:
(a) providing an alkane-containing feed;
(b) providing at least one active material within a reaction zone, wherein the active material comprises i) a first component having alkane dehydrogenation functionality and ii) a second component having selective hydrogen combustion functionality and oxygen storage functionality;
(c) during a first time interval,
i. flowing the alkane-containing feed to the reaction zone,
ii. catalytically dehydrogenating at least a portion of the alkane, in the presence of the first component, to produce a reaction mixture comprising olefin and molecular hydrogen, wherein the reaction zone is within a temperature range of from 400° C. to 700° C.,
iii. selectively combusting, in the presence of the second component, at least a portion of the second reaction mixture's molecular hydrogen to produce the olefin-rich product,
iv. transferring heat from the reaction zone, during the catalytic dehydrogenation of the alkane and the selective combustion of the molecular hydrogen to maintain a variance of not greater than 10° C. from the average temperature of the reaction zone during the catalytic dehydrogenation of the alkane and the selective combustion of the hydrogen, and
v. removing the olefin-rich product from the reaction zone;
(d) curtailing or ceasing the flow of the alkane-containing feed to the reaction zone;
(e) during a second time interval,
i. flowing an oxygen-containing fluid to the reaction zone, and
ii. storing at least a portion of the oxygen with the second component; and
(f) reducing or ceasing the flow of the oxygen-containing feed to the reaction zone.

22. The catalytic alkane dehydrogenation reaction process of claim 21, wherein the active material includes the second component in the amount ≧10 wt. % based on the weight of the active material, based on total volume of the catalyst material.

23. The catalytic alkane dehydrogenation reaction process of claim 21, wherein the first component is a chromium-based dehydrogenation catalyst, and the oxygen-containing fluid flowed to the reaction zone in step (e) comprises greater than 15 mole % oxygen, based on total amount of oxygen-containing fluid flowed to the reaction zone.

24. The catalytic alkane dehydrogenation reaction process of claim 21 wherein, steps (c) through (0 are repeated in successive time intervals.

25. An apparatus for catalytically dehydrogenating an alkane-containing feed to produce an olefin-containing product, the apparatus comprising:
(a) a reactor vessel having an interior volume;
(b) at least one reaction zone within the reactor vessel's interior volume, wherein the reaction zone includes at least one bed of active material comprising dehydrogenation catalyst;
(c) at least one inlet conduit in fluidic communication with the reactor vessel's interior volume for conveying the alkane containing feed into the reaction zone;
(d) at least one heat transfer conduit for bringing a heat transfer fluid into indirect thermal contact with the reaction zone, wherein the heat transfer conduit is
(i) substantially closed to flow of the heat transfer fluid into the reactor vessel's interior volume,
(ii) substantially open to the flow of heat between the reaction zone and the heat transfer fluid, and
(iii) configured to maintain the reaction zone in a substantially isothermal temperature profile at an average temperature 400° C. to 700° C. during the catalytic dehydrogenation; and
(e) at least one outlet conduit in fluidic communication with the reactor vessel's interior volume for conveying the olefin containing product away from reaction zone.
